(19) United States
(12) Patent Application Publication
(10) Pub. No.: US 2024/0342558 A1
(43) Pub. Date: Oct. 17, 2024

(54) OUTDOOR SWIMMING TRAJECTORY DETERMINATION METHOD, SYSTEM, APPARATUS, AND STORAGE MEDIUM

(71) Applicant: GUANGDONG COROS SPORTS TECHNOLOGY CO., LTD, Dongguan (CN)

(72) Inventors: Xuan RAO, Dongguan (CN); Xiaofu XIA, Dongguan (CN); Haotian NIU, Dongguan (CN)

(21) Appl. No.: 18/757,468

(22) Filed: Jun. 27, 2024

Related U.S. Application Data
(63) Continuation of application No. PCT/CN2022/102120, filed on Jun. 29, 2022.

(51) Int. Cl. A63B 24/00

(52) U.S. Cl. CPC A63B 24/0075; A63B 24/0021; A63B 2220/12; A63B 2220/30; A63B 2220/40; A63B 2220/62; A63B 2220/836; A63B 2244/20

(57) ABSTRACT

An outdoor swimming trajectory determination method includes in a situation where positioning data is not obtained, obtaining a swimming direction of a user and a swimming acceleration of the user, the swimming direction being collected by a first terminal, determining a swimming trajectory of the user according to the swimming direction and the swimming acceleration, and in a situation where the positioning data is obtained, correcting the swimming trajectory according to the positioning data, The swimming acceleration is collected by at least one of the first terminal or a second terminal. The positioning data is collected by the second terminal.

### CROSS-REFERENCE TO RELATED APPLICATION

This application is a continuation of International Application No. PCT/CN2022/102120, filed Jun. 29, 2022, the entire content of which is incorporated herein by reference.

### TECHNICAL FIELD

The present disclosure generally relates to the smart wearable apparatus technology field and, more particularly, to an outdoor swimming trajectory determination method, an outdoor swimming trajectory determination system, an outdoor swimming trajectory determination apparatus, and a storage medium.

### BACKGROUND

GPS-based sports tracking apparatuses become popular among athletes and sports enthusiasts and are broadly applied in different sports to perform data recording. A sports tracking apparatus can monitor a user geographic position in real-time by receiving global positioning information to obtain a real time speed of the user to further form a user movement trajectory.

In outdoor swimming, to avoid affecting swimming techniques, the user usually fixes the sports tracking apparatus with a positioning function at an arm of the user. Since the arm is under water for a long time during swimming, a satellite signal cannot be transmitted through the water and received by the sports tracking apparatus. Thus, the swimming activity of the user cannot be monitored through the sports tracking apparatus. For example, the swimming trajectory cannot be accurately recorded. The application of the sports tracking apparatus is limited in the swimming activity.

For the problem that the swimming trajectory cannot be continuously obtained, the existing technology provides a solution of fixing a movement sensor on the arm of the user and performing the recognition on the swimming trajectory of the user through the movement sensor. Another solution can include fixing the sports tracking apparatus with the positioning function on the arm of the user and obtaining the positioning signal when the arm coming out of the water during swimming to record the swimming trajectory. However, a speed cumulative error of the movement sensor increases with time. Meanwhile, the movement sensor can be affected by the instability of the arm swing. Thus, the determined movement trajectory can have a big error compared to the actual movement trajectory, which is difficult to satisfy the actual needs of the user. The solution of determining the swimming trajectory through the positioning apparatus can only be suitable for a stroke in which the arm is often or regularly swung out of the water and is not suitable for other types of strokes. Moreover, the accuracy cannot be ensured for the positioning signal obtained within a short period, which greatly reduces the accuracy of the determined movement trajectory.

### SUMMARY

In accordance with the disclosure, there is provided an outdoor swimming trajectory determination method. The method includes in a situation where positioning data is not obtained, obtaining a swimming direction of a user and a swimming acceleration of the user, the swimming direction being collected by a first terminal, determining a swimming trajectory of the user according to the swimming direction and the swimming acceleration, and in a situation where the positioning data is obtained, correcting the swimming trajectory according to the positioning data. The swimming acceleration is collected by at least one of the first terminal or a second terminal. The positioning data is collected by the second terminal.

In accordance with the disclosure, there is provided an outdoor swimming trajectory determination system, including a first terminal and a second terminal. The first terminal and the second terminal are configured to be communicatively connected to each other. The first terminal is configured to collect a swimming direction of a user and send the swimming direction to the second terminal. The second terminal is configured to collect a swimming acceleration of the user and determine a swimming trajectory of the user according to the swimming acceleration and the swimming direction. The second terminal is further configured to collect positioning data and correct the swimming trajectory according to the positioning data.

In accordance with the disclosure, there is provided an outdoor swimming trajectory determination system, including a first terminal and a second terminal. The first terminal and the second terminal are configured to be communicatively connected to each other. The first terminal is configured to collect a swimming direction and a swimming acceleration of a user and determine a swimming trajectory of the user according to the swimming acceleration and the swimming direction. The second terminal is configured to collect positioning data and send the positioning data to the first terminal. The first terminal is further configured to correct the swimming trajectory according to the positioning data.

### BRIEF DESCRIPTION OF THE DRAWINGS

FIG. 1 is a schematic flowchart of an outdoor swimming trajectory determination method according to some embodiments of the present disclosure.

FIG. 2 is a schematic flowchart of another outdoor swimming trajectory determination method according to some embodiments of the present disclosure.

FIG. 3 is a schematic flowchart showing determining a swimming speed according to a swimming acceleration according to some embodiments of the present disclosure.

FIG. 4 is a schematic flowchart showing determining a user swimming trajectory according to a swimming acceleration and a swimming direction according to some embodiments of the present disclosure.

FIG. 5 is a schematic flowchart showing correcting a swimming trajectory of positioning data according to some embodiments of the present disclosure.

FIG. 6 is a schematic structural diagram of an outdoor swimming trajectory determination system according to some embodiments of the present disclosure.

FIG. 7 is a schematic structural diagram of another outdoor swimming trajectory determination system according to some embodiments of the present disclosure.

FIG. 8 is a schematic structural diagram of an outdoor swimming trajectory determination apparatus according to some embodiments of the present disclosure.

### DETAILED DESCRIPTION OF THE EMBODIMENTS

The technical solutions of embodiments of the present disclosure are described in connection with the accompanying drawings of embodiments of the present disclosure. Described embodiments are merely some embodiments of the present disclosure.

In embodiments of the present disclosure, the terms “first,” “second,” etc., are used to distinguish between similar objects, rather than to describe a specific order or sequence. Data used in this way can be interchangeable. Thus, embodiments of the present disclosure described here can be implemented in a sequence other than those sequences shown or described here. In addition, the terms “including,” “containing,” and any variations thereof are intended to cover non-exclusive inclusions. For example, a process, method, system, product, or apparatus that includes a series of steps or units is not necessarily limited to those clearly listed steps or units, but can include other steps or units not listed or inherent to the process, method, product, or apparatus.

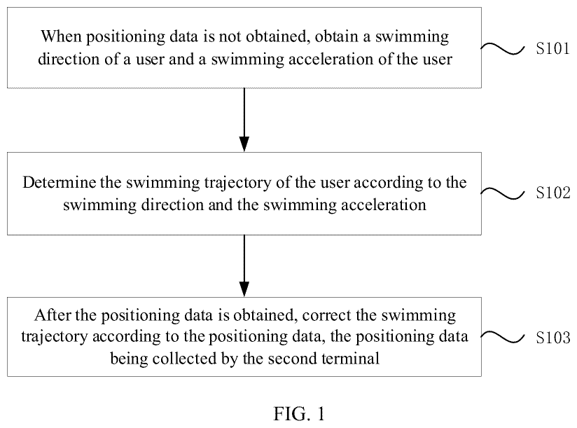

FIG. 1 is a schematic flowchart of an outdoor swimming trajectory determination method according to some embodiments of the present disclosure. The method can be performed by an outdoor swimming trajectory determination system formed by a first terminal and a second terminal arranged at different positions of a user body. The outdoor swimming trajectory determination system can be implemented by software and/or hardware and can be integrated in an outdoor swimming trajectory determination apparatus. As shown in FIG. 1, the method includes the following processes.

At S101, in a situation where positioning data is not obtained, a swimming direction and a swimming acceleration of the user are obtained.

The swimming direction can be collected by the first terminal, and the swimming acceleration can be collected by the first terminal and/or the second terminal.

In some embodiments, the positioning data can be determined based on a global positioning system, and the current position of the user can be accurately determined according to the received satellite positioning signal. The swimming direction can refer to a swimming orientation during swimming of the user who wears the first terminal and the second terminal. That is, heading information under the ground coordinate system during the swimming of the user. The swimming acceleration can refer to acceleration information generated by differences in swimming effort, stroke amplitude, and stroke frequency during the swimming of the user and can reflect swimming status information such as the speed of the user.

In some embodiments, the first terminal can be a terminal apparatus that is worn by the user on the body and has a plurality of types of movement sensors for collecting the swimming direction and the swimming acceleration of the user during outdoor swimming. The second terminal can be a terminal apparatus that is worn by the user on the body and has a plurality of types of movement sensors and positioning function for collecting the swimming acceleration and the positioning data of the user during the outdoor swimming. In some embodiments, the second terminal can be a smartwatch, which is not limited in embodiments of the present disclosure.

In some embodiments, the first terminal can be arranged at the head or torso of the user, and the second terminal can be arranged on the arm of the user. Since good direction stability is required to collect the swimming direction of the user, the orientation of the head and torso of the user can be consistent with the swimming direction for most of time during swimming. That is, the direction stability of the head or torso of the user can be considered to be good. Thus, the first terminal can be arranged at the head or torso of the user. Since information transmission is performed between the first terminal and the second terminal, the distance between the second terminal and the first terminal may not be too long. Moreover, the first terminal can be always above the water or at a shallow position under the water, and the second terminal can be configured to collect the positioning data during the swimming of the user. If the user maintains the second terminal above the water, for example, the user can wear the first terminal and the second terminal at the same position, the second terminal can collect the positioning data in real-time and can consume a lot of electrical energy. Thus, the second terminal can be normally arranged at the arm of the user. Thus, the second terminal can communicate with the first terminal under a short distance and may not need to always obtain the positioning data, and the consumption of the electrical energy can be reduced.

When the positioning data is not obtained, the second terminal may not often be out of the water or may not be maintained out of the water for a long time. Thus, the swimming trajectory of the user cannot be formed according to the positioning information obtained by the second terminal. Then, the swimming direction of the user can be collected by the first terminal fixed at the head or torso of the user. The first terminal or the second terminal arranged at the arm of the user can collect the swimming acceleration to reflect the swimming status of the user.

In some embodiments, the first terminal can include a nine-axis sensor, i.e., a three-axis gyroscope, a three-axis acceleration sensor, and a three-axis magnetometer sensor. Since the nine-axis sensor measures sensor data under the user coordinate system, the roll angle and the pitch angle information may need to be calculated when the heading information is calculated to project the data collected by the three-axis magnetometer into the ground coordinate system.

In some embodiments, determining the swimming direction can include performing integration on the original angular speed data obtained by the three-axis gyroscope sensor to obtain the initial posture information of the user. The initial posture information can include an initial roll angle, an initial pitch angle, and an initial heading angle. The initial roll angle and the initial pitch angle can be tilted angles of the user as a carrier relative to the horizontal ground surface. The initial heading angle can be an included angle between the orientation of the user as the carrier and a geographical north pole. Meanwhile, since the three-axis gyroscope sensor includes error factors such as zero-bias, proportional factor, and three circle non-orthogonal, the angle obtained by performing integration cumulative summation through the original angular speed data can have an increasing error as time flies. Thus, the angle data determined by the three-axis gyroscope sensor may need to be calibrated through the three-axis acceleration sensor and the three-axis magnetometer sensor. The acceleration information collected by the three-axis acceleration sensor when the three-axis acceleration sensor is relatively static in a period of time. Since in a regular movement process, the data collected by the three-axis acceleration sensor can periodically fluctuate around the gravity acceleration vector. Thus, the direction corresponding to the acceleration information collected by the three-axis acceleration sensor can be the direction of the earth gravity. The initial roll angle and the initial pitch angle can be calibrated with the determined earth gravity direction to obtain a calibrated roll angle and a calibrated pitch angle. Since the initial heading angle is sensor data under the user coordinate system, the initial heading angle can be projected onto the ground coordinate system through the calibrated pitch angle and the calibrated roll angle. The initial heading angle can be calibrated according to the heading information collected by the three-axis magnetometer sensor. Then, the direction corresponding to the heading information obtained by calibrating the initial heading angle can be determined as the swimming direction of the user.

At S102, the swimming trajectory of the user is determined according to the swimming direction and the swimming acceleration.

In some embodiments, the swimming trajectory can be a movement trajectory formed by connecting positioning points determined according to the swimming direction, the swimming acceleration, and a predetermined collection interval and can be used to indicate positions through which the user swims by.

When the current position, the current swimming direction, and the swimming acceleration of the user are known, if the predetermined collection interval is provided, the distance that the user swims in the current swimming direction with the swimming acceleration during the predetermined collection interval. Then, the predicted position where the user should be relative to the current position can be determined after the predetermined collection interval. The current position and the predicted position can be connected. Thus, the swimming trajectory from the current collection moment to a next collection moment of the user can be obtained. Then, the next collection moment can be used as the new current collection moment to repeat the above operation. That is, the swimming trajectory can be determined according to the swimming acceleration and the swimming direction obtained during swimming of the user.

At S103, after the positioning data is obtained, the swimming trajectory is corrected according to the positioning data. The positioning data is obtained by the second terminal.

Before starting to swim, the second terminal can obtain the accurate positioning data before the user enters the water to indicate the starting position where the user starts to swim. During swimming, the accurate satellite positioning data cannot be obtained during the determination of the swimming trajectory. The determination of the swimming trajectory can be affected by the deviation and cumulative error of the sensor. Thus, the determined swimming trajectory can have a certain difference from the actual swimming trajectory. After the accurate positioning data is obtained by the second terminal, the position corresponding to the positioning data can be the accurate position of the user at the current moment. Thus, the swimming trajectory from the last positioning data acquisition moment to the current positioning data acquisition moment can be corrected according to the accurate position.

The solution of determining the outdoor swimming trajectory of embodiments of the present disclosure can be applied to the outdoor swimming trajectory determination system. The outdoor swimming trajectory determination system can include the first terminal and the second terminal arranged at different positions of the user body. When the positioning data is not obtained, the swimming direction and the swimming acceleration of the user can be obtained. The swimming direction can be collected by the first terminal, and the swimming acceleration can be collected by the first terminal and/or the second terminal. The swimming trajectory of the user can be determined according to the swimming direction and the swimming acceleration. After obtaining the positioning data, the swimming trajectory can be corrected according to the positioning data. The positioning data can be obtained by the second terminal. By adopting the technical solution, and through the first terminal and the second terminal arranged at different positions of the user body, the swimming direction and the swimming acceleration of the user can be collected when the positioning data is not obtained. Then, the swimming trajectory of the user can be determined according to the swimming direction and the swimming acceleration without the positioning data. After obtaining the positioning data, the determined swimming trajectory can be corrected according to the positioning data. Thus, the swimming trajectory can be determined when the positioning data cannot be obtained in real-time. Meanwhile, the determined swimming trajectory can be corrected through the positioning data that is obtained intermittently, which satisfies the determination of the swimming trajectory of the user under a plurality of strokes, and improves the accuracy of determining the outdoor swimming trajectory.

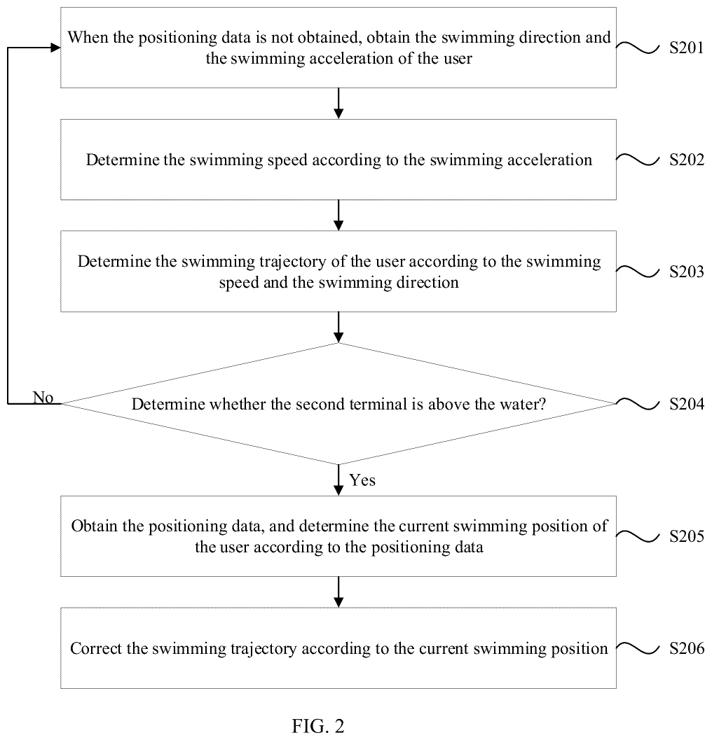

FIG. 2 is a schematic flowchart of another outdoor swimming trajectory determination method according to some embodiments of the present disclosure. The method includes the following processes.

At S201, in a situation where the positioning data is not obtained, the swimming direction and the swimming acceleration of the user are obtained.

The swimming direction can be collected by the first terminal, and the swimming acceleration can be collected by the first terminal and/or the second terminal.

At S202, the swimming speed is determined according to the swimming acceleration.

In some embodiments, the swimming speed can be a real time speed of the user in the swimming direction at the current moment.

When the swimming acceleration of the user is known, and the starting position of the swimming is identified, the real time speed corresponding to the current moment can be determined according to the swimming acceleration and kinematic equations. The real time speed can be determined as the swimming speed of the user. The present disclosure only provides a solution of determining the swimming speed according to the swimming acceleration and does not limit the determination method of the swimming speed.

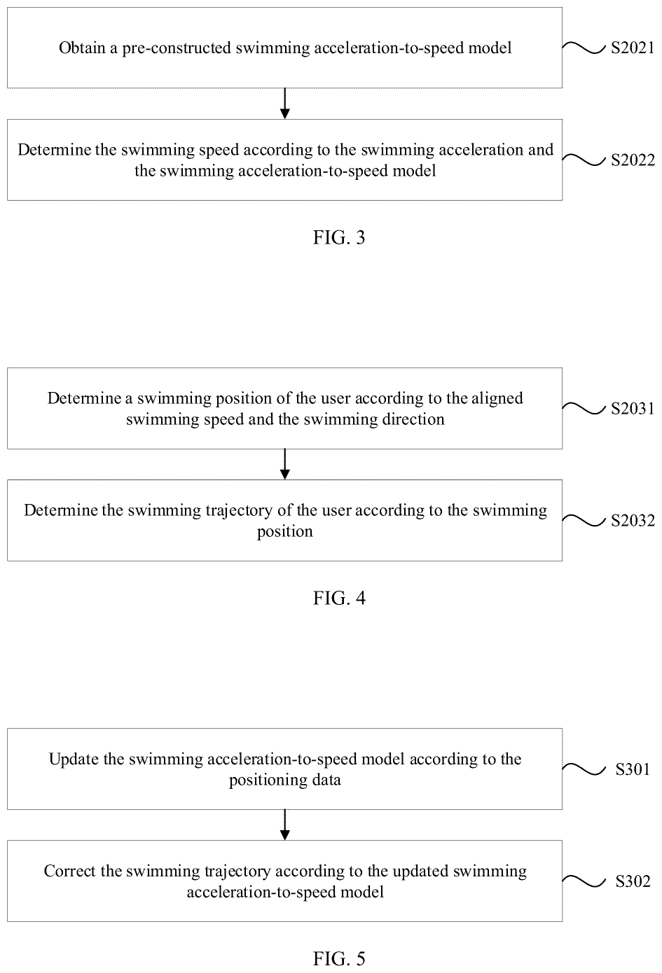

FIG. 3 is a schematic flowchart showing determining a swimming speed according to a swimming acceleration according to some embodiments of the present disclosure. Determining the swimming speed according to the swimming acceleration includes the following processes.

At S2021, a pre-constructed swimming acceleration-to-speed model is obtained.

In some embodiments, the swimming acceleration-to-speed model can be a mathematical model that is pre-constructed according to the actual needs and configured to represent the relationship between the swimming acceleration and the swimming speed. In some embodiments, the swimming acceleration-to-speed model can be a table format or another adaptive setting format, which is not limited in embodiments of the present disclosure.

In some embodiments, constructing the swimming acceleration-to-speed model can include collecting three-axis acceleration data corresponding to each person swimming a fixed distance with a uniform speed under each stroke from a group of test people with a predetermined number, dividing the collected three-axis acceleration data into a plurality of windows according to a predetermined time length, and extracting acceleration feature information from each window. The extracted acceleration feature information can include peak size, peak count, peak interval, variance, and mean. Information such as the hand stroke frequency, above and under water time, and stroke amplitude of the corresponding stroke of the test person can be determined according to the extracted acceleration feature information. Mapping relationships between the swimming accelerations and the speeds can be constructed under different strokes. Then, the corresponding swimming acceleration-to-speed model that is suitable for all users can be obtained.

At S2022, the swimming speed is determined according to the swimming acceleration and the swimming acceleration-to-speed model.

When the stroke of the user is known, the swimming acceleration-to-speed model corresponding to the stroke of the user can be determined. The swimming speed corresponding to the swimming acceleration can be determined by performing table lookup in the swimming acceleration-to-speed model according to the swimming acceleration.

At S203, the swimming trajectory of the user is determined according to the swimming speed and the swimming direction.

When the swimming speed and the swimming direction of the user are known, a swimming distance within a period of time can be determined in the swimming direction for the user according to the speed and time equation. Then, the position reached by the user after swimming the corresponding distance in the swimming direction relative to the current position can be determined after a period of time. The connection line between the position and the current position can be referred to as the swimming trajectory of the user in the period of time.

Before determining the swimming trajectory of the user according to the swimming speed and the swimming direction, the method can also include obtaining a timestamp of the outdoor swimming trajectory determination system and aligning the swimming speed and swimming direction according to the timestamp.

In some embodiments, the timestamp can be data generated using the digital signature technology, which can be used to indicate collection time of different data in the outdoor swimming trajectory determination system.

The timestamp of the outdoor swimming trajectory determination system can be obtained. The timestamp can include timestamps corresponding to the swimming speed and the swimming direction in the outdoor swimming trajectory determination system, respectively. The timestamps corresponding to the swimming speed and the swimming direction in the outdoor swimming trajectory determination system, respectively, can be used to indicate the collection time of the swimming speed and the swimming direction in the outdoor swimming trajectory determination system. According to the timestamp corresponding to the swimming speed and the timestamp corresponding to the swimming direction, the swimming speed and the swimming direction collected at the same time can be aligned. Thus, the swimming information of the user at the time corresponding to the timestamp can be represented by the aligned swimming speed and the swimming direction.

In some embodiments, the time alignment can be performed on the first terminal and the second terminal in the outdoor swimming trajectory determination system before the user swims. The time corresponding to the initial timestamp can be represented by TO. After the user starts swimming, the first terminal and the second terminal can perform time accumulation based on the initial timestamp. When the swimming direction and the swimming acceleration are obtained, the corresponding timestamp can be carried by the swimming direction and the swimming acceleration according to the acquisition time. Since the obtained swimming acceleration carries the timestamp, the swimming speed that is determined according to the swimming acceleration can also carry the timestamp corresponding to the swimming acceleration. Based on the timestamps carried by the swimming speed and the swimming direction, the swimming speed and the swimming direction can be aligned. That is, the real time swimming direction and the real time swimming speed can be determined during the swimming of the user.

FIG. 4 is a schematic flowchart showing determining a user swimming trajectory according to a swimming acceleration and a swimming direction according to some embodiments of the present disclosure. Determining the user swimming trajectory includes the following processes.

At S2031, a swimming position of the user is determined according to the aligned swimming speed and the swimming direction.

The swimming speed and the swimming direction can be aligned. That is, the swimming speed and the swimming direction can correspond to the speed and direction of the user at the same moment during swimming. Based on the swimming speed, the swimming distance of the user between the current timestamp and the next timestamp can be determined. According to the swimming direction, a predicted position of the user at the moment corresponding to the next timestamp relative to the current position can be determined. The predicted position can be determined as the swimming position of the user corresponding to the next timestamp.

At S2032, the swimming trajectory of the user is determined according to the swimming position.

By connecting swimming positions of a plurality of consecutive timestamps, the swimming trajectory from the initial moment when the user starts swimming to the moment corresponding to each timestamp of the plurality of consecutive timestamps can be obtained.

At S204, whether the second terminal is above the water is determined. If the second terminal is above the water, proceed to S205, and if the second terminal is not above the water, return to S201.

The second terminal can have the positioning function, which is usually implemented by receiving the satellite positioning signal. When the second terminal is under the water, the blockage of the water can affect the positioning precision of the positioning information obtained by the second terminal, and maintaining the positioning function on for a long time can consume power of the second terminal. Thus, whether the second terminal is above the water is determined after determining the swimming trajectory. If the second terminal is above the water, the position of the user can be precisely positioned through the second terminal, and S205 can be performed. If the second terminal is not above the water, the second terminal still cannot perform the positioning on the position of the user precisely, and the method can return to S201 to continue to record the swimming trajectory of the user according to the swimming direction and the swimming acceleration.

At S205, the positioning data is obtained, and the current swimming position of the user is determined according to the positioning data.

The positioning data can be received by the second terminal and can be the satellite positioning signal received by the second terminal. Then, the position determined by the satellite positioning signal can be used as the current swimming position of the user at the current moment.

At S206, the swimming trajectory is corrected according to the current swimming position.

When the current swimming position is not consistent with the position corresponding to the current moment in the swimming trajectory, the current swimming position determined according to the positioning data can be considered as the real position of the user at the current moment. Then, the swimming trajectory from the acquisition of the last piece of positioning data to the acquisition time of the positioning data corresponding to the current swimming position can be used as a to-be-corrected swimming trajectory. The to-be-corrected swimming trajectory can be fit-corrected according to the current swimming position.

In some embodiments, a fit-correction method can include rotation, stretching, reverse accumulation of the to-be-corrected swimming trajectory from the current swimming position, and weight average, which is not limited in embodiments of the present disclosure.

FIG. 5 is a schematic flowchart showing correcting the swimming trajectory according to the positioning data according to some embodiments of the present disclosure. Correcting the swimming trajectory according to the positioning data includes the following processes.

At S301, the swimming acceleration-to-speed model is updated according to the positioning data.

The timestamp alignment can be performed on the second terminal and the first terminal before the user swims. According to the positioning data obtained by the second terminal and the last piece of positioning data obtained by the second terminal, the distance and average speed information can be determined between two pieces of positioning data. The speed information can be considered as the speed generated under the current stroke and the current swimming acceleration of the user. Then, the mapping relationship between the swimming acceleration and the swimming speed can be adjusted in the swimming acceleration-to-speed model to update the swimming acceleration-to-speed model. For example, the swimming acceleration-to-speed model can be updated each time the positioning data is obtained to cause the updated swimming acceleration-to-speed model to be more adaptive to determining the current swimming status of the user.

At S302, the swimming trajectory is corrected according to the updated swimming acceleration-to-speed model.

The obtained swimming acceleration can be substituted into the updated swimming acceleration-to-speed model to determine the updated swimming speed. Then, the swimming trajectory can be updated and corrected according to the time length corresponding to two neighboring timestamps, the updated swimming speed, and the swimming direction.

In the outdoor swimming trajectory determination solution of embodiments of the present disclosure, through the pre-constructed swimming acceleration-to-speed model, the swimming speed corresponding to the swimming acceleration of the user that is obtained in real time can be determined. Then, by configuring the timestamp corresponding to the outdoor swimming trajectory determination system for the first terminal and the second terminal, the information obtained by the first terminal and the second terminal can be aligned and matched. According to the aligned swimming speed and the swimming direction, the swimming trajectory can be determined when the positioning data is not obtained. Thus, the accuracy of determining the swimming trajectory without positioning data can be improved. Meanwhile, the positioning data can only be obtained when the second terminal is above the water to avoid ineffectively obtaining the positioning data for a long time to save the power of the second terminal. After obtaining the positioning data, the swimming trajectory can be directly corrected according to the positioning data. The swimming trajectory can also be corrected after correcting the swimming acceleration-to-speed model according to the positioning data. Since the swimming acceleration-to-speed model is a model matching the stroke of the user, the swimming acceleration-to-speed model can satisfy the determination of the swimming trajectory under a plurality of strokes of the user and the acquisition requirement of the positioning data. Thus, the accuracy of determining the outdoor swimming trajectory can be improved.

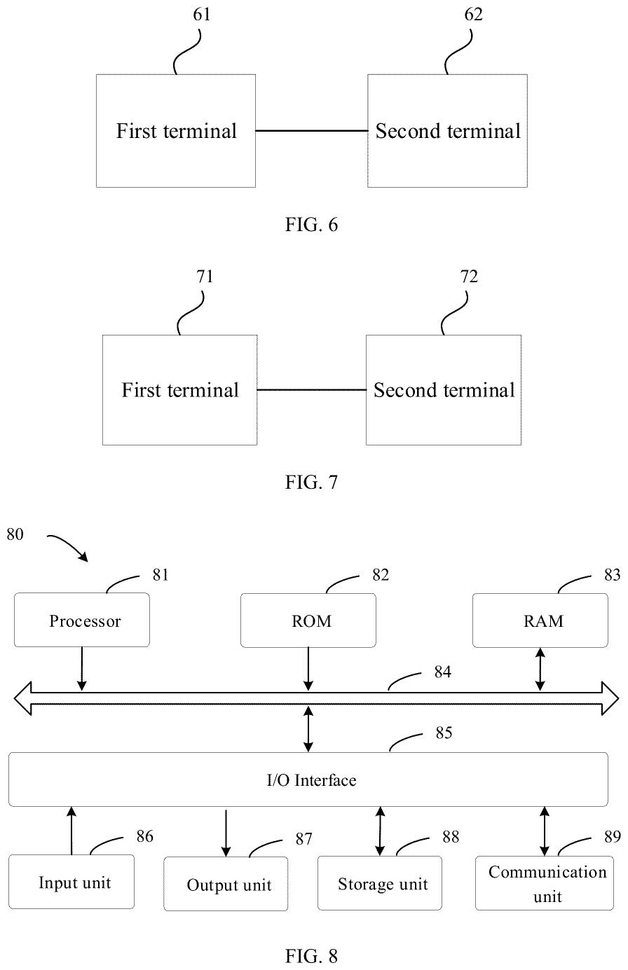

FIG. 6 is a schematic structural diagram of an outdoor swimming trajectory determination system according to some embodiments of the present disclosure. As shown in FIG. 6, the system includes a first terminal 61 and a second terminal 62.

The first terminal 61 can be arranged at the head or torso of the user. The second terminal 62 can be arranged at the arm of the user. The first terminal 61 and the second terminal 62 can be communicatively connected.

The first terminal 61 can be configured to collect the swimming direction of the user and send the swimming direction to the second terminal 62. The second terminal 62 can be configured to collect the swimming acceleration of the user and determine the swimming trajectory of the user according to the swimming acceleration and the swimming direction. The second terminal 62 can be further configured to collect the positioning data and correct the swimming trajectory according to the positioning data.

In the technical solution of embodiments of the present disclosure, the swimming trajectory can be determined when the positioning data cannot be obtained in real-time, and the determined swimming trajectory can be corrected through the positioning data that is obtained intermittently. Thus, the determination of the swimming trajectory under the plurality of strokes of the user can be satisfied, and the accuracy of the determination of the swimming trajectory of the user can be improved.

The outdoor swimming trajectory determination system of embodiments of the present disclosure can execute the outdoor swimming trajectory determination method of embodiments of the present disclosure, and have the corresponding functional modules and effects of executing the outdoor swimming trajectory determination method.

FIG. 7 is a schematic structural diagram of another outdoor swimming trajectory determination system according to some embodiments of the present disclosure. As shown in FIG. 7, the system includes a first terminal 71 and a second terminal 72.

The first terminal 71 can be arranged at the head or torso of the user. The second terminal 72 can be arranged at the arm of the user. The first terminal 71 and the second terminal 72 can be communicatively connected.

The first terminal 71 can be configured to collect the swimming direction and the swimming acceleration of the user and determine the swimming trajectory of the user according to the swimming acceleration and the swimming direction. The second terminal 72 can be configured to collect the positioning data and send the positioning data to the first terminal 71 when the positioning data is collected. The first terminal 71 can be further configured to correct the swimming trajectory according to the positioning data.

In the technical solution of embodiments of the present disclosure, the swimming trajectory can be determined when the positioning data cannot be obtained in real time, and the determined swimming trajectory can be corrected through the positioning data that is obtained intermittently. Thus, the determination of the swimming trajectory under the plurality of strokes of the user can be satisfied, and the accuracy of the determination of the swimming trajectory of the user can be improved.

The outdoor swimming trajectory determination system of embodiments of the present disclosure can execute the outdoor swimming trajectory determination method of embodiments of the present disclosure, and have the corresponding functional modules and effects of executing the outdoor swimming trajectory determination method.

FIG. 8 is a schematic structural diagram of an outdoor swimming trajectory determination apparatus 80 according to some embodiments of the present disclosure. The outdoor swimming trajectory determination apparatus 80 includes the first terminal or the second terminal in the outdoor swimming trajectory determination system. The outdoor swimming trajectory determination apparatus 80 can be an electronic apparatus including a plurality of forms of digital computers, such as a laptop computer, a desktop computer, a workstation, a personal digital assistant, a server, a blade server, a large-scale computer, and any other appropriate computers. The electronic apparatus can include a plurality of forms of mobile devices, such as a personal digital processor, a cellular phone, a smartphone, a wearable apparatus (e.g., a helmet, glasses, and a watch), and any other similar computing devices. Members, connections and relationships of the members, and functions of the members shown in the present disclosure are merely exemplary and are not intended to limit the present disclosure described and/or required in the present specification.

As shown in FIG. 8, the outdoor swimming trajectory determination apparatus 80 includes at least one processor 81 and at least one memory communicatively connected to the at least one processor 81, such as a Read-Only Memory (ROM) 82, and a Random Access Memory (RAM) 83. The memory can store a computer program that can be executed by the at least one processor. The processor 81 be configured to execute a plurality of appropriate actions and processing according to the computer program stored in ROM 82 or the computer program loaded in RAM 83 from a storage nit 88. RAM 83 can also store a plurality of programs and data required by the operation of the outdoor swimming trajectory determination apparatus 80. The processor 81, the ROM 82, and the RAM 83 can be connected to each other through a bus 84. An input/output (I/O) interface 85 is also connected to the bus 84.

A plurality of members in the outdoor swimming trajectory determination apparatus 80 can be connected to the I/O interface 85, including a input unit 86, such as a keyboard and a mouse, an output unit 87, such as a plurality of types of monitors and a speaker, a storage unit 88, such as magnetic disks and optical disc, and a communication unit 89, such as a network card, a modern, and a wireless communication transceiver. The communication unit 89 can allow the outdoor swimming trajectory determination apparatus 80 to exchange information/data with other apparatuses through a computer network such as Internet and/or a plurality of types of communication networks.

The processor 81 can include a plurality of types of general purpose and/or special purpose processing assembly having a processing and computation capability. In some embodiments, the processor 81 can include but is not limited to a central processing unit (CPU), a graphics processing unit (GPU), a plurality of types of special purpose artificial intelligence (AI) computation chips, a plurality of types of processors of executing the machine learning model algorithms, a digital signal processor (DSP), and any propriate processors, controllers, and micro-controllers. The processor 81 can be configured to execute the methods and processes of embodiments of the present disclosure, e.g., the outdoor swimming trajectory determination method.

In some embodiments, the outdoor swimming trajectory determination method can be implemented as a computer program, which can be tangibly contained in a computer-readable storage medium such as storage unit 88. In some embodiments, all or a part of the computer program can be loaded and/or installed onto the outdoor swimming track determination apparatus 80 via ROM 82 and/or communication unit 89. When the computer program is loaded into RAM 83 and executed by the processor 81, one or more processes of the above outdoor swimming track determination method can be executed. In some other embodiments, the processor 81 can be configured to execute the outdoor swimming trajectory determination method in any suitable methods (e.g., using firmware).

The implementations of the systems and technologies described above can be implemented in digital electronic circuit system, integrated circuit system, Field-Programmable Gate Array (FPGA), Application-Specific Integrated Circuit (ASIC), Application-Specific Standard Part (ASSP), System on Chip (SoC), Complex Programmable Logic Device (CPLD), computer hardware, firmware, software, and/or combinations thereof. The implementations can include the systems and technologies being implemented in one or more computer programs. The one or more computer programs can be executed and/or explained in a programmable system including the at least one programmable processors. The programmable processors can be special purpose or general purpose programmable processors, which can receive data and instructions from a storage system, at least one input device, and at least one output device and can transmit the data and instruction to the storage system, the at least one input device, and the at least one output device.

The computer program used to implement the method of the present disclosure can be programmed in one or more programming languages. These computer programs can be provided to the general purpose computer, the special purpose computer, or processors of other programmable digital data processing devices to implement the functions/operations defined in the flowchart and/or block diagram when the computer programs are executed by the processor. The computer programs can be wholly executed on the machine, partially executed on the machine, partially executed on the machine as an independent software package and partially executed on a remote machine or wholly executed on the remote machine.

In the context of the present disclosure, the computer-readable storage medium can be a tangible medium including or storing the computer programs for the instruction execution system, device, or apparatus to or for use in connection with the instruction execution system, device, or apparatus. The computer-readable storage medium can include but is not limited to an electronic, magnetic, optical, electromagnetic, infrared, or semiconductor system, device, or apparatus, or any suitable combination thereof. In some other embodiments, the computer-readable storage medium can be a machine-readable signal medium. For example, the machine-readable storage medium can include electrical connections based on one or more wires, portable computer disks, hard drives, RAM, ROM, Erasable Programmable Read-Only Memory (EPROM or flash memory), optical fiber, portable Compact Disc Read-Only Memory (CD-ROM), optical storage apparatuses, magnetic storage apparatuses, or any suitable combinations thereof.

To provide user interaction, the systems and technologies described here can be implemented on the electronic apparatus. The electronic apparatus can include a display device (e.g., Cathode Ray Tube (CRT) or Liquid Crystal Display (LCD) monitor) configured to display information for the user, and a keyboard and a pointing device (e.g., a mouse or a track ball). The user can provide inputs to the electronic apparatus through the keyboard and the pointing device. Other types of devices can be configured to provide the user interaction. For example, the feedback provided to the user can include any types of sensory feedback (e.g., visual feedback, auditory feedback, or tactile feedback), and the inputs from the user can be received in any forms (including sound input, voice input, or tactile input).

The systems and technologies described here can be implemented in a computation system including a back-end member (e.g., a data server), a computation system including a middleware member (e.g., an application server), a computation system including a front-end member (e.g., a user computer having a graphical user interface or a web browser, the user can interact with the implementations of the systems and technologies through the graphic user interface or the network browser), or a combination thereof. The members of the system can be connected through digital data communications (e.g., communication networks) of any types or media. The communication networks can include local area networks (LANs), wide area networks (WANs), blockchain networks, and Internet.

The computing system can include a client and a server. The client and the server are generally away from each other and interact with each other through the communication networks. The client-server relationship can be generated by executing the computer program having the client-server relationship on the corresponding computer. The server can be a cloud server, or can be also referred to as a cloud computing server or a cloud host, which is a host product in the cloud computing service system. Thus, disadvantages such as high management difficulty and poor business scalability in the existing physical host and virtual private server (VPS) can be solved.

The plurality of types of processes can be used to rearrange, add, or delete processes. For example, the plurality of processes of the present disclosure can be executed in parallel, sequentially, or in different sequences, as long as the expected result of the technical solution can be achieved, which is not limited here.

What is claimed is:

1. An outdoor swimming trajectory determination method comprising:
in a situation where positioning data is not obtained, obtaining a swimming direction of a user and a swimming acceleration of the user, the swimming direction being collected by a first terminal, and the swimming acceleration being collected by at least one of the first terminal or a second terminal;
determining a swimming trajectory of the user according to the swimming direction and the swimming acceleration; and
in a situation where the positioning data is obtained, correcting the swimming trajectory according to the positioning data, the positioning data being collected by the second terminal.

2. The method according to claim 1, wherein determining the swimming trajectory includes:
determining a swimming speed according to the swimming acceleration; and
determining the swimming trajectory according to the swimming speed and the swimming direction.

3. The method according to claim 2, further comprising, before determining the swimming trajectory:
obtaining a timestamp of a system implementing the method; and
aligning the swimming speed and the swimming direction according to the timestamp;
wherein determining the swimming trajectory includes:
determining a swimming position of the user according to the swimming speed and the swimming direction that are aligned; and
determining the swimming trajectory of the user according to the swimming position.

4. The method according to claim 2, wherein determining the swimming speed includes:
obtaining a pre-constructed swimming acceleration-to-speed model; and
determining the swimming speed according to the swimming acceleration and the swimming acceleration-to-speed model.

5. The method according to claim 4, wherein correcting the swimming trajectory according to the positioning data includes:
updating the swimming acceleration-to-speed model according to the positioning data to obtain an updated swimming acceleration-to-speed model; and
correcting the swimming trajectory according to the updated swimming acceleration-to-speed model.

6. The method according to claim 1, wherein correcting the swimming trajectory includes:
determining a current swimming position of the user according to the positioning data; and
performing fit-correction on the swimming trajectory according to the current swimming position.

7. The method according to claim 6, wherein a fit-correction method includes rotation and stretching.

8. The method according to claim 1, further comprising, after determining the swimming trajectory:
obtaining the positioning data in response to detecting that the second terminal is above water.

9. The method according to claim 1, wherein the first terminal is arranged at a head or torso of the user, and the second terminal is arranged at an arm of the user.

10. An outdoor swimming trajectory determination apparatus comprising:
one or more processors; and
one or more memories connected to the one or more processors and storing one or more computer programs that, when executed by the one or more processors, cause the one or more processors to perform the method of claim 1.

11. The apparatus according to claim 10, wherein the one or more computer programs, when executed by the one or more processors, further cause the one or more processors to:
determine a swimming speed according to the swimming acceleration; and
determine the swimming trajectory according to the swimming speed and the swimming direction.

12. The apparatus according to claim 11, wherein the one or more processors are further configured to:
obtain a timestamp of a system implementing the method; and
align the swimming speed and the swimming direction according to the timestamp;
determine a swimming position of the user according to the swimming speed and the swimming direction that are aligned; and
determine the swimming trajectory of the user according to the swimming position.

13. The apparatus according to claim 11, wherein the one or more processors are further configured to:
obtain a pre-constructed swimming acceleration-to-speed model; and
determine the swimming speed according to the swimming acceleration and the swimming acceleration-to-speed model.

14. The apparatus according to claim 13, wherein the one or more processors are further configured to:
update the swimming acceleration-to-speed model according to the positioning data to obtain an updated swimming acceleration-to-speed model; and
correct the swimming trajectory according to the updated swimming acceleration-to-speed model.

15. The apparatus according to claim 10, wherein the one or more processors are further configured to:
determine a current swimming position of the user according to the positioning data; and
perform fit-correction on the swimming trajectory according to the current swimming position.

16. The apparatus according to claim 15, wherein a fit-correction method includes rotation and stretching.

17. The apparatus according to claim 10, wherein the one or more processors are further configured to:
obtain the positioning data in response to detecting that the second terminal is above water.

18. A non-transitory computer-readable storage medium storing one or more computer programs that, when executed by one or more processors, cause the one or more processors to perform the method of claim 1.

19. An outdoor swimming trajectory determination system, comprising a first terminal and a second terminal, wherein:
the first terminal and the second terminal are configured to be communicatively connected to each other;
the first terminal is configured to collect a swimming direction of a user and send the swimming direction to the second terminal;
the second terminal is configured to collect a swimming acceleration of the user and determine a swimming trajectory of the user according to the swimming acceleration and the swimming direction; and
the second terminal is further configured to collect positioning data and correct the swimming trajectory according to the positioning data.

20. An outdoor swimming trajectory determination system, comprising a first terminal and a second terminal, wherein:
the first terminal and the second terminal are configured to be communicatively connected to each other;
the first terminal is configured to collect a swimming direction and a swimming acceleration of a user and determine a swimming trajectory of the user according to the swimming acceleration and the swimming direction;
the second terminal is configured to collect positioning data and send the positioning data to the first terminal; and
the first terminal is further configured to correct the swimming trajectory according to the positioning data.
